(19) United States
(12) Patent Application Publication
(10) Pub. No.: US 2006/0024753 A1
(43) Pub. Date: Feb. 2, 2006

(54) Immunoglobulin-positive neurons in Alzheimer disease are dying via the classical, antibody-dependent, complement pathway

(76) Inventor: Michael R. D'Andrea, Cherry Hill, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/188,479

(22) Filed: Jul. 25, 2005

Related U.S. Application Data
(60) Provisional application No. 60/591,479, filed on Jul. 27, 2004.

(51) Int. Cl. G01N 33/53; G01N 33/567

(52) U.S. Cl. 435/7.1; 435/7.2

(57) ABSTRACT

Ig-positive neurons, which have been shown to be present in Alzheimer's disease, are shown to have complement C1q and C5b-9 proteins. C1q and C5b-9 can be employed in diagnosis and treatment of Alzheimer's disease.

### CROSS-REFERENCE TO RELATED APPLICATIONS

This application claims priority to Application No. 60/591,479 filed on Jul. 27, 2004.

### BACKGROUND OF THE INVENTION

Investigations into the causes of Alzheimer's disease (AD) range from vascular pathology and neuroinflammation to neuropathologic components such as amyloid plaques and neurofibrillary tangles. However, it may appear that AD may actually represent a conglomerate of several simultaneous or sequential normal and pathologic processes. Although it seems possible that AD may have a single, primary cause, this initial insult remains elusive. Recently, in comparison to age-matched, non-demented control brain tissues, significant increases of detectable parenchymal immunoglobulins in AD brain tissues were reported and most importantly, dramatic increases of Ig-positive neurons were observed (1). Furthermore, a significant number of these Ig-positive neurons showed neurodegenerative apoptotic features that were rarely observed in Ig-negative neurons (1). These data implied a critical link between a faulty blood-brain barrier (BBB) and neuronal death through an autoimmune mechanism (1, 2). However, little is known about these Ig-positive neurons.

### SUMMARY OF THE INVENTION

The inflammatory profile of Ig-positive neurons have been characterized. Specifically, it has been determined that the complement products, C1q, a specific component of the classical, antibody-induced, complement pathway (3), and C5b-9, which represents the membrane attack complex (4), were co-localized with the Ig-positive neurons. The association of reactive microglia with these Ig-positive neurons has also been characterized and it was determined that the positive neurons were preferentially associated or targeted by reactive microglia in comparison to Ig-negative neurons.

### BRIEF DESCRIPTION OF THE DRAWINGS

FIG. 1. Representative examples of pan-Ig (A), C1q (B) and C5b-9 (C) immunolabeling in several pyramidal neurons (arrows) of the AD entorhinal cortex among neurons without pan-Ig, C1q or C5b-9 detection (arrowheads). Bar=50 μm (insert, 25 μm).

FIG. 2. Representative serially-sectioned (5 mm) sets show the co-localization of pan-Ig and C1q (A, B, C) and the co-localization of pan-Ig and C5b-9 (D) immunolabeling (arrows) in several neurons of the AD entorhinal cortex among neurons without prominent pan-Ig, C1q or C5b-9 detection (arrowheads). Asterisks show areas of serial vessels. Bar=100 μm (A); 25 μm (insert A); 50 μm (B-D); D=12 μm (insert D).

FIG. 3. Representative double IHC images (A-H) show the presence of red-labeled, HLA-DR-positive, reactive microglia in contact or in association with brown-labeled, Ig-positive neurons (arrows) in the AD entorhinal cortex. Arrowheads show the presence of nearby Ig-negative neurons without associated reactive microglia. Bar=25 μms.

FIG. 4. Graphed data shows that the HLA-DR immunolabeled reactive microglia tend to be significantly closer to the Ig-positive neurons (n=101; 7.69 μm±0.72 SE) than the Ig-negative neurons (n=172; 31.74 μm±1.44 SE) in representative areas (n=10, see text) of the AD (n=5) entorhinal cortex. Mann-Whitney Rank Sum Test (*p<0.001).

### DETAILED DESCRIPTION OF THE INVENTION

Postmortem entorhinal cortical brain tissues from patients with sporadic AD (n=12) and age-matched control (n=6) were obtained from the Harvard Brain Tissue Resource Center (HBTRC, Belmont, Mass., USA) and fixed in 10% neutral-buffered formalin. Pathological confirmation of AD included (1) the presence of amyloid plaques, (2) the presence of neurofibrillary tangles and (3) reduced neuronal density (1, 5, 6). Tissues were trimmed and processed for paraffin embedding according to conventional methods. Five-micron sections were serially cut, mounted onto SuperFrost Plus⁺ (Fisher Scientific, Pittsburgh, Pa.) microscopic slides and dried overnight. The protocols for routine single immunohistochemistry (IHC) have been described in detail previously (1, 5, 6). Briefly, tissue sections on microscopic slides were dewaxed and re-hydrated. Slides were microwaved in Target buffer (Dako, Carpenturia, Calif.), cooled, placed in phosphate-buffered saline (pH 7.4, PBS) and treated with 3.0% H₂O₂ for 10 min at room temperature. All incubations (30 min each) and washes were performed at room temperature. Normal blocking serum (Vector Labs, Burlingame, Calif.) was placed on all slides for 10 min. After a brief rinse in PBS, sections were treated with the primary antibodies: polyclonal anti-human pan-Ig recognizes IgG, IgM, IgA (1:1000, Serotec, N.C.; Dako, Calif.); polyclonal anti-human C1q (1:200, Quidel, Calif.); monoclonal anti-human C5b-9 (1:200, Quidel, Calif.); and monoclonal anti-human HLA-DR, a marker of reactive microglia (3, 4, 6, 7, 8) (1:200, Chemicon, Temecula, Calif.). Slides were then washed in PBS and treated with goat anti-rabbit (polyclonal primaries) or horse anti-mouse (monoclonal primaries) biotinylated secondary antibodies (Vector Labs). After washing in PBS, the avidin-biotin-horseradish peroxidase complex reagent (ABChp, Vector Labs) was added. All slides were washed and treated with 3,3′-diaminobenzidine (DAB, Biomeda, Foster City, Calif.) 2 times 5 min, rinsed in distilled water, and counterstained with hematoxylin.

In addition, to determine if there was a spatial (two-dimensional) association between the reactive microglia and the Ig-positive neurons, the distance (μms) between the nuclei of Ig-positive or Ig-negative neurons and prominent, red-labeled, HLA-DR immunoreactive microglial fibers were measured using image analysis (Image Pro, ver. 4.01, Phase III Imaging, Glen Mills, Pa.). To accomplish this objective, double IHC was applied. Briefly, the first primary antibody was detected using the ABC-alkaline phosphotase (ABCap) reagent followed by the Fast Red (Sigma, Mo.) chromogen, while the second primary antibody (pan-Ig) was detected using the ABChp-DAB system (1, 6, 8). Negative controls included replacement of the primary antibody with the antibody diluent or pre-absorption of the primary antibody with its specific antigen as previously demonstrated for the Ig antibodies (1). Ig-positive and Ig-negative neurons (average 20/tissue) were analyzed in 10 random fields (40× objective)/tissue (n=5). Statistical analyses were performed using the Mann-Whitney Rank Sum Test using SigmaStat and plotted using SigmaPlot.

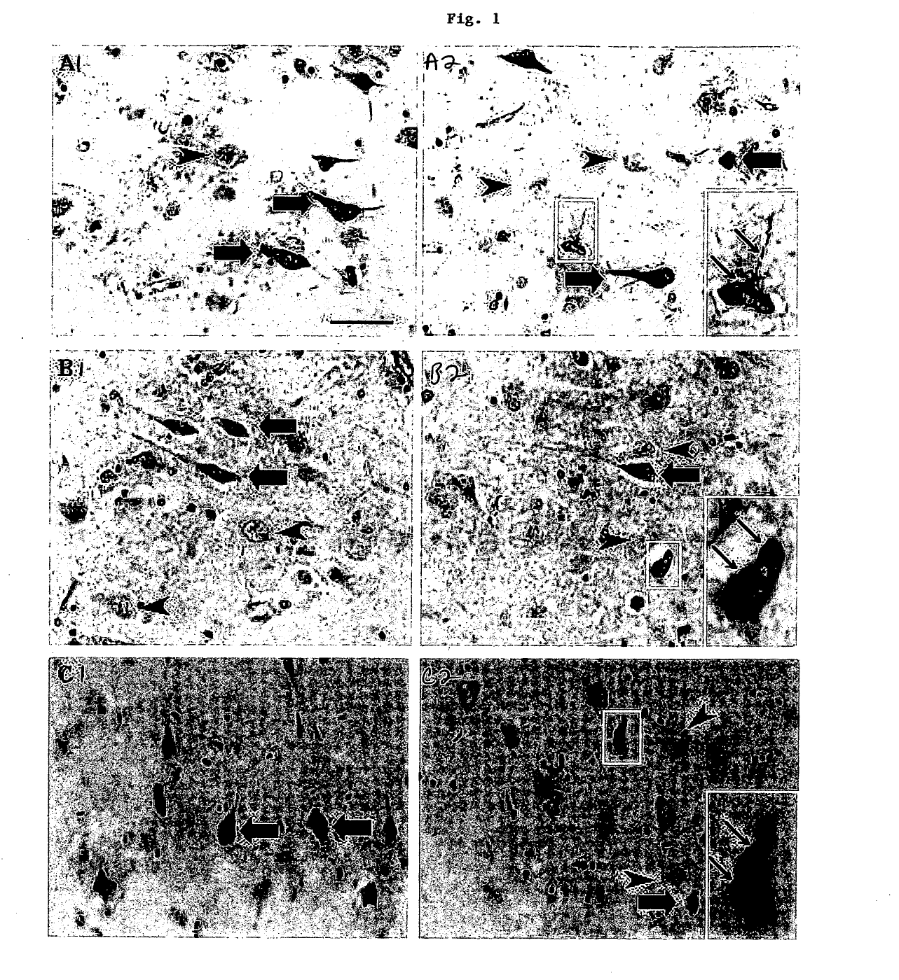

Representative brown labeled, Ig-, C1q- and C5b-9-immunopositive neurons (arrows) were detected in the entorhinal cortex of AD brain tissues (FIG. 1). Not detectable immunolabeling was observed in the negative controls (data not presented). The panels in FIG. 1A show the presence of intensely immunolabeled Ig-positive neurons (arrows) within close proximity (within microns) of Ig-negative neurons (arrowheads), which had also been previously reported (1, 2). Note the neurodegenerative features such as cell atrophy and dense, pyknotic nuclear chromatin (FIG. 1A, insert) of the Ig-positive neurons (arrows, insert) amidst normally appearing Ig-negative neurons with prominent nucleoli embedded in normally appearing, transcriptional active nuclear euchromatin (arrowheads), which was also previously described (1, 2).

The panels in FIG. 1B show prominent C1q-immunopositive neurons (arrows) amidst C1q-negative neurons (arrowheads). This labeling was primarily located in the neuronal perikaryon with diminished signal in the somatic-dendritic area. It was interesting to note that these C1q-positive neurons exhibited the same neurodegenerative features as the Ig-positive neurons with the atrophic perikaryon and dense, pyknotic nuclear chromatin (FIG. 1B, insert) suggesting not only the C1q-positive neurons were degenerative but that the Ig and C1q immunolabeling patterns may be co-localized in neurons. Weak C1q immunolabeling was also detected in some morphologically healthy neurons indicating basal expression detection, as the expression of C1q in another study was low in naïve mice but increased in experimental autoimmune encephalomyelitis (EAE) (9).

Similar findings were also observed in the C5b-9-immunopositive neurons (arrows, FIG. 1C). Again, only a small population of the neurons was C5b-9 immunolabeled, some of which appeared neurodegenerative (FIG. 1C, insert). Arrowheads in FIG. 1C show the lack of C5b-9 detection in neighboring neurons.

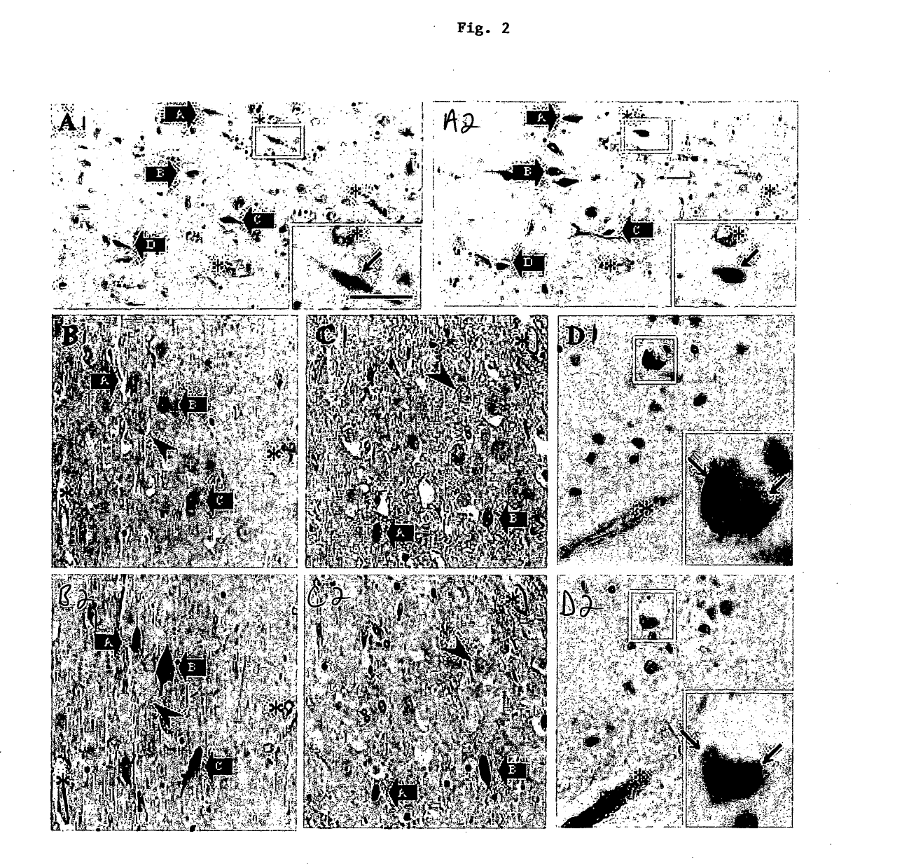

Even though the labeling patterns of the Ig, C1q and C5b-9 appeared to be associated with neurodegenerative neurons, it was not clear if they were also present in the same cell until we analyzed serially (5 μs) sectioned labeled tissues. These data showed that C1q and C5b-9 are indeed present in Ig-positive neurons (FIG. 2). Specifically, in representative serial sets, FIGS. 2A-C showed C1q immunoreactivity (FIG. 2A-C) in the Ig-positive neurons. C5b-9 immunoreactivity was also detected in the Ig-positive neurons (FIG. 2D). However, in this particular example, relatively weaker Ig immunolabeling was detected (FIG. 2D insert) in the C5b-9 neuron, which may suggest the lost of Ig immunoreactivity as the cell nears death. Importantly, the data obtained from these assays also showed that Ig-positive neurons are neurodegenerative and are C1q and C5b-9 immunopositive suggesting a strong association between these signatures or profiles.

As noted, age-matched control brains were also analyzed for the presence of Ig, C1q and C5b-9. Although parenchymal Ig immunolabeling was detected in these tissues, most of the labeling was restricted around large vessels (data not presented), as previously described (1). Several Ig-positive neurons were only observed in one of the six age-matched control brain tissues (data not presented), which also showed C1q and C5b-9 immunolabeling patterns.

C1q, a classical complement pathway component (3, 10) (FIG. 1B), and C5b-9, a marker of the terminal step in the complement pathway and representing the membrane attack complex (4, 10, 11) (FIG. 1C) were detected in Ig-positive neurons (FIG. 2) providing evidence for the presence of the classical, antibody-dependent (not alternative, antibody-independent) complement pathway of cell death to the Ig-positive neurons. Immunocytochemical studies have demonstrated that the complement cascade is fully activated in the AD hippocampus and neocortex resulting in ‘deposition’ of C5b-9 on dystrophic neuritis, NFTs and senile plaques (12, 13), and now within Ig-positive neuronal perikaryon. The significance of intracellular Ig, C1q and C5b-9 is not known but may represent internalization as well as de novo synthesis (14). As a note, complement activation products have been described as deposition material (15, 16). However, any evidence of ‘deposition’ from a random point of view was not observed. To clarify, any detection of complement components were associated with cells in this study.

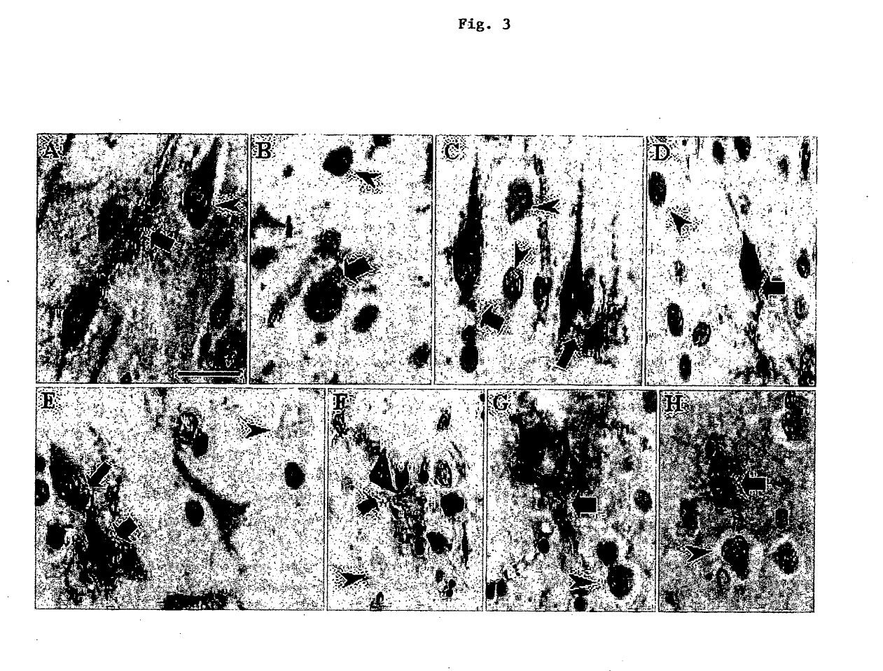

Next, a series of assays were performed to characterize the spatial distribution of the reactive microglia (red-labeled) with Ig-positive neurons (brown-labeled) using double IHC on tissue sections (FIG. 3). In FIG. 3A-E, reactive microglia are located within very close proximity (possibly in contact) to many Ig-positive neurons (arrows), which were not readily observed nearby Ig-negative neurons (arrowheads). Although subject to over interpretation, the microglia detected in FIG. 3A may represent an early stage of discovery as the Ig-positive neuron appears morphologically healthy, as compared to the microglia detected in FIG. 3 panels B-D appear in contact with the Ig-positive neuronal perikaryon that appear slightly dystrophic. The arrangement of the microglia and Ig-positive neurons in FIGS. 3E-G resemble that of a war zone (late stage) where several red-labeled, reactive microglia appear dramatically engaged with the degenerative Ig-positive neurons (FIGS. 3E, F, G). Note the lack of associated reactive microglia with the Ig-negative neurons (arrowheads, FIG. 3).

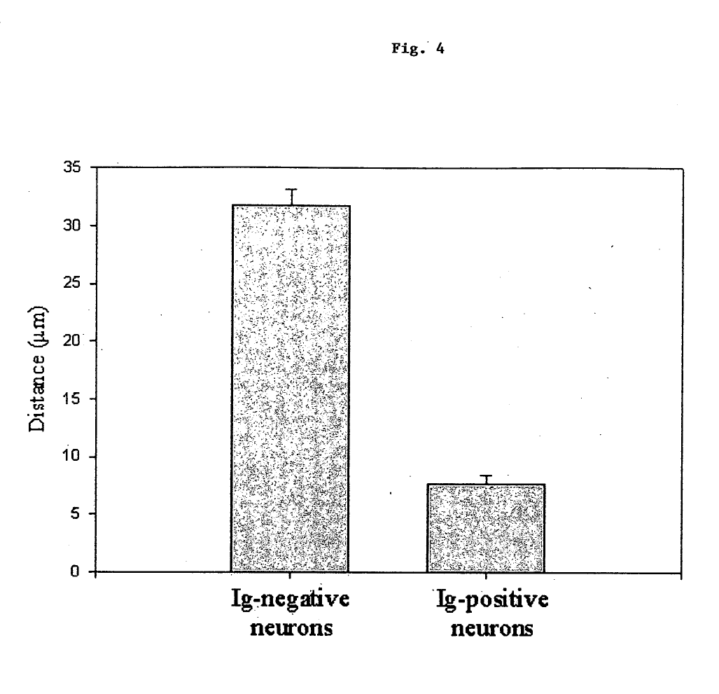

In an effort to determine if the reactive microglia target Ig-positive neurons over the Ig-negative cells, the average distances between the HLA-DR-positive processes of the microglia to the Ig-positive and Ig-negative neuronal nuclei was measured using image analysis. As presented in FIG. 4, the analytical data showed that the reactive microglia are significantly (p<0.001) more associated with the Ig-positive neurons than the Ig-negative neurons. Although these data were obtained from static, two-dimensional images, it is difficult to ignore the possibility that reactive microglia are more associated with the Ig-positive (n=101; mean=7.69 μm±0.72 SE) that the Ig-negative (n=172; mean=31.74 μm±1.44 SE) neurons.

These data suggest the involvement of microglia in the pending cell death process. In support, C1q and C5b-9 can bind to the surface of apoptotic cells resulting in the phagocytosis of these cells by microglia (17, 18). Also, complement produced locally by reactive microglia was activated on the membranes of neurons in Huntington's disease contributing to neuronal necrosis as well as proinflammatory activities (14). Hence, it is logical to explain the presence of these microglia on these Ig-positive complement-ridden neurons, but it remains to be determined if the glial cells are recruited to these neurons or if they contribute to complement accumulation on these neurons. However, a recent study suggested the former to be true, as there was significantly less microglial activation surrounding fibrillar Aβ deposits in the C1q null mouse (7).

Previously, it was proposed that the presence of the antibody-induced classical complement pathway did not exist in AD because the discovery of an antibody remained “unequivocally demonstrated” in spite of overwhelming evidence of classical pathway components and activation fragments reported in the AD brain (13, 19, 20). Subsequently, β-amyloid as well as other neuropathological markers had been proposed to be responsible for complement activation (21). As a personal observation, Aβ42 was detected in Ig-positive and Ig-negative neurons suggesting that Ig immunoreactivity was independent of Aβ42 immunoreactivity in neurons (data not presented).

An autoimmune AD hypothesis based upon the dramatic increases of vascular-derived, parenchymal Igs and Ig-positive, neurodegenerative neurons had been suggested (1, 2). The data from the present study validates and extends the proposed pathway (1,2) which most likely begins with a dysfunctional, unregulated, BBB that allows non-discriminatory passage of vascular derived-Ig into the brain parenchyma. Once in the CNS, some of these once benign antibodies, inconsequentially bind to their neuronal or neuronal-like antigen(s) on neurons leading to complement formation (MAC) resulting in an autoimmune/classical (antibody-dependent) complement-cell death process in the AD brain. These observations are not limited to AD, as the activation of the classical complement system is also known to play a central role in autoimmune demylineation (9), and in multiple sclerosis, myasthenia gravis, head trauma and stroke (22). Although not clear, the ability or capacity of these vascular derived antibodies to specifically or non-specifically bind to their neuronal target may able be dependent upon its isotype, avidity and affinity (1,2) as well as its ability to fix complement. Each factor will play an important role in determining the ‘clinical’ pathogenicity of an autoantibody response to its antigen (20) and may explain why BBB breach alone, or that the presence of parenchymal Igs may not always lead to AD. Regardless, the effects of the “inconsequential” binding of these antibodies to a specific population of neurons did not appear favorable.

The data suggests that populations of Ig-positive neurons are dying via the classical complement pathway and that microglia are preferentially associated with many of these degenerating neurons. Unfortunately, once the 2-hit (presence of specific neuronal damaging antibodies and BBB breach) cascade pathway begins, subsequent processes of inflammation could inflict additional neuronal cell death (23) independent of immunoglobulin and complement. As it was previously noted (1, 2), the presence of this “auto”-antibody, once characterized, should provide a new therapeutic target to treat and possibly prevent AD. In the meantime, therapeutic opportunities should be designed to preserve the integrity of the BBB in an effort to block the anomalous presence and subsequent deleterious actions of autoantibody(s) into the CNS, while other strategies could be directed to augment, remove or block the autoantibody while it is in the vascular system before it gains circuitous entry into the CNS. Minimally, in the context of this work, CNS imaging data to assess BBB integrity “coupled” with the presence of vascular disease indicators and autoimmune products (i.e. complement) could provide diagnostic and prognostic capabilities, in addition to the clinical cognitive testing paradigms.

### REFERENCES

1. D'Andrea M R: Evidence linking neuronal cell death to autoimmunity in Alzheimer's disease. Brain Research 2003; 982: 19-30.
2. D'Andrea MR: Add Alzheimer's disease to the list of autoimmune diseases. Medical Hypotheses 2005; 64(3): 458-463.
3. Emmerling M R, Watson M D, Ruby C A, Spiegel K: The role of complement in Alzheimer's disease pathology. Biochem Biophys Acta 2000; 1502: 158-171.
4. Webster S, Lue L F, Brachova L, Tenner A J, McGeer P L, Teria K, Walker D G, Bradt B, Cooper N R, Rogers J: Molecular and cellular characterization in the membrane attack complex, C5b-9, in Alzheimer's disease. Neurobiology of Aging 1997; 18: 415-421.
5. D'Andrea MR, Nagele R G, Wang H-Y, Peterson P A, Lee D H S: Evidence that neurons accumulating amyloid can undergo lysis to form amyloid plaques in Alzheimer's disease. Histopathology 2001; 38: 120-134.
6. D'Andrea M R, Reiser P A, Gumula N A, Hertzog B M, Andrade-Gordon P: Application of triple-label immunohistochemistry to characterize inflammation in Alzheimer's disease brains. Biotechnic & Histochemistry 2001; 76: 97-106.
7. Fonseca M I, Zhou J, Botto M, Tenner A J: Absence of C1q leads to less neuropathology in transgenic mouse models of Alzheimer's disease. J Neuroscience 2004; 24(29): 6457-6465.
8. Wang HY, Lee DHS, D'Andrea MR, Peterson P A, Shank R, Reitz A: β-amyloid₁₋₄₂ binds to α7 nicotinic acetylcholine receptor with high affinity: Implications for Alzheimer's disease pathology. J Biol Chem 2000; 275(8): 5626-5632.
9. Weerth S H, Rus H, Shin M L, Raine S H: Complement C5 in experimental autoimmune encephalomyelitis (EAE) facilitates remyelination and prevents gliosis. Am J Pathol 2003; 163(3): 1069-1080.
10. Pasinetti G M: Inflammatory mechanisms in neurodegeneration and Alzheimer's disease: The role of the complement system. Neurobiol of Aging 1996; 17(5): 707-716.
11. Rogers J, Cooper NR, Schultz J, McGeer P L, Webster S, Styren S D, Civin W H, Brachova L, Bradt B, Ward P, Lieberburg I: Complement activation by β-amyloid in Alzheimer's disease. Proc Natl Acad Sci USA 1992; 89: 10016-10020.
12. Loeffler D A, Camp D M, Schonberger M B, Singer D J, LeWitt P A: Early complement activation increases in the brain in some aged normal subjects. Neurobio of Aging 2004; 25(8): 1001-1007.
13. Shen Y, Lue L-F, Yang L-B, Roher A, Kuo Y-M, Strohmeyer R, Goux W J, Lee V, Johnson G V W, Webster S D, Cooper N R, Bradt B, Rogers J: Complement activation by neurofibrillary tangles in Alzheimer's disease. Neurosci Letters 2001; 305: 165-168.
14. Singhrao S K, Neal J W, Morgan B P, Gasque P: Increased complement biosynthesis by microglia and complement activation on neurons in Huntington's disease. Exp Neurology 1999; 159: 362-376.
15. Barnum S R: Complement biosynthesis in the central nervous system. Crit Rev Oral Biol Med 1995; 6: 132-146.
16. Mukherjee P, Pasinetti G M: The role of complement anaphylatoxin C5a in neurodegeneration: implications in Alzheimer's disease. J Neuroimmunology 2000; 105: 124-130.
17. Korb L C, Ahearn J M: C1q binding directly and specifically to surface belbs of aopototic human keratinocytes. J Immunol 1997; 158: 4525-4528.
18. Webster S D, Yang A J, Margol L, Garzon-Rodriguez W, Glabe CG, Tenner A J: Complement component C1q modulates the phagocytic behavior of microglia. Exp Neurol 2000; 161: 127-138.
19. Neuroinflammation Working Group, Inflammation and Alzheimer's Disease. Neurobiol of Aging 2000; 21: 383-421.
20. Piddlesden S J, Lassmann H, Zimprich F, Morgan B P, Linington L: The demyelinating potential of autoantibodies to myelin oligodendrocyte glycoprotein is related to their ability to fix complement. Am J Pathol 1993; 143(2): 555-564.
21. Velazquez P, Cribbs D H, Poulos T L, Tenner A J: Aspartate residue 7 in amyloid β-protein is critical for classical complement pathway activation: Implications for Alzheimer's disease pathogenesis. Nature Medicine 1997; 3(1): 77-79.
22. Spiegel K, Barnum S R, Emmerling M L in: P. L. Wood (Ed.), Neuroinflammation: Mechanisms and Management, Human Press, pp. Totowa, N.J., 1997, 129-176.
23. D'Andrea MR, Cole GM, Ard M D: The microglial phagocytic role with specific plaque types in the Alzheimer disease brain. Neurobiol of Aging 2004; 25(5): 675-683.

1. A method to diagnose the presence of Alzheimer's Disease in a subject by detecting increased immunoglobulin-positive neurons in said subject as compared to normal controls, wherein said increased immunoglobulin-positive neurons are detected as follows:
extracting brain tissue of said subject;
assaying said extract for immunoglobulin-positive neurons; and
comparing said immunoglobulin-positive neurons exhibited in the same assay by similar extracts from brain tissue of controls.

2. A method to diagnose the presence of Alzheimer's Disease in a subject by detecting increased C1q protein in said subject as compared to normal controls, wherein said increased C1q protein is detected as follows:
extracting brain tissue of said subject;
assaying said extract for C1q protein; and
comparing said C1q protein exhibited in the same assay by similar extracts from brain tissue of controls.

3. A method to diagnose the presence of Alzheimer's Disease in a subject by detecting increased C5b-9 protein in said subject as compared to normal controls, wherein said increased C5b-9 protein is detected as follows:
extracting brain tissue of said subject;
assaying said extract for C5b-9 protein; and
comparing said C5b-9 protein exhibited in the same assay by similar extracts from brain tissue of controls.

4. The method of claim 1, wherein said method is conducted post mortem.

5. The method of claim 2, wherein said method is conducted post mortem.

6. The method of claim 3, wherein said method is conducted post mortem.
